(19) United States
(12) Patent Application Publication
(10) Pub. No.: US 2026/0151644 A1
(43) Pub. Date: Jun. 4, 2026

(54) A Wearable Device for Promoting Hair Growth Through Phototherapy

(71) Applicant: Shenzhen Kaiyan Medical Equipment Co., Ltd, Shenzhen (CN)

(72) Inventors: Alain Dijkstra, Amstelveen (NL); Tongming Chen, Shenzhen (CN); Li Xiang, Shenzhen (CN); Yin Zhichao, Shenzhen (CN); Cao Jiaming, Shenzhen (CN)

(21) Appl. No.: 18/968,060

(22) Filed: Dec. 4, 2024

(51) Int. Cl. A61N 5/06

(52) U.S. Cl. CPC A61N 5/0617; A61N 2005/0626; A61N 2005/0647; A61N 2005/0652; A61N 2005/0663

(57) ABSTRACT

The present invention relates to a wearable device (100) for promoting hair growth through phototherapy. The wearable device (100) includes a wearing body (10) to be used to wear on a head, a treatment pad (20) configured inside the wearing body (10), and a control box (30) arranged on the wearing body (10). The treatment pad (20) is provided to emit light onto the user's scalp, stimulating hair growth through phototherapy. The control box (30) is detachably attached to the wearing body (10) through the treatment pad (20). The treatment pad (20) is having a connecting portion (25) that extends beyond the wearing body (10) and is adapted to allow the control box (30) to attach to the treatment pad (20) establishing an electrical connection with the treatment pad (20) and enabling the device to function effectively.

### TECHNICAL FIELD

The present invention relates to a wearable device. More specifically, the present invention relates to a wearable device for promoting hair growth through phototherapy.

### BACKGROUND

Presently, hair loss is a widespread concern affecting millions of people globally. Whether due to stress, overwork, genetics, or other health-related factors, many individuals experience thinning hair or even baldness, which can have a significant impact on their appearance and self-confidence. Hair is often closely tied to how we present ourselves, and for those struggling with hair loss, it can lead to anxiety, reduced self-esteem, and social discomfort. As a result, there is a growing demand for effective hair restoration solutions that are easy to use, accessible, and deliver results.

In response to this demand, various technologies have been developed to promote hair growth, with phototherapy emerging as a popular and promising method. Phototherapy treatments, which use low-level laser or LED (Light-emitting diode) light, work by stimulating the scalp and hair follicles, increasing blood circulation and encouraging hair growth. This non-invasive method has proven to be effective, as it helps to improve hair density and strength without the need for drugs or surgery. However, the current equipment used for phototherapy-based hair growth has several limitations.

Traditionally, large phototherapy machines have been used in clinics and specialized treatment centers to address hair loss. While these machines are effective, they come with significant drawbacks. They are often large, expensive, and require professional operation, making them impractical for personal, at-home use. Most individuals cannot afford these treatments, and even those who can may find the machines too bulky and inconvenient for regular use.

To solve this problem, the market has introduced portable phototherapy caps for at-home use. These caps are designed to provide the same benefits as the larger machines, but in a more accessible, wearable format. However, the current models still fall short in several ways. Many of these caps have complicated designs, making them difficult to clean and maintain. Additionally, they often feature built-in batteries within the cap itself, which makes charging and disassembly cumbersome. This design not only complicates their usability but also introduces safety concerns, such as the risk of overheating during use or improper charging.

Therefore, there is a need for a wearable device for promoting hair growth through phototherapy to overcome a few or all drawbacks of the existing technologies.

### STATEMENT OF THE INVENTION

An object of the present invention is to provide a wearable device for promoting hair growth through phototherapy.

Another object of the present invention is to provide a wearable device for promoting hair growth through phototherapy that easily connects and disconnects from the wearable device, simplifying the process of charging, maintenance, and cleaning, while enhancing the device's safety and usability.

Another object of the present invention is to provide a wearable device for promoting hair growth through phototherapy that is aesthetically appealing, portable, and suitable for everyday use, allowing users to conveniently and effectively treat hair loss without the need for expensive, clinic-based treatments.

Yet another object of the present invention is to provide a wearable device for promoting hair growth through phototherapy that establishes electrical connections between the control box, the treatment pad, and the wearing body, ensuring secure attachment, ease of use, and enhanced safety during operation.

Another object of the present invention is to provide a wearable device for promoting hair growth through phototherapy to enhance the flexibility and adaptability of the device, allowing for better fit and customization to different head shapes and sizes.

One more object of the present invention is to provide a wearable device for promoting hair growth through phototherapy that provides easily accessible control and energy storage unit without affecting user comfort, and providing an efficient way to adjust and control the treatment.

According to the present invention, there is provided a wearable device for promoting hair growth through phototherapy. The wearable device for promoting hair growth through phototherapy may include a wearing body provided to be worn on a user's head, a treatment pad, and a control box. The treatment pad may be arranged to configure an inner shape of the wearing body and may be adapted to emit light onto the user's scalp. The control box may be attachable to the wearing body, with the treatment pad having a connecting portion that extends beyond the wearing body, allowing the control box to be attached and establish an electrical connection with the treatment pad.

The treatment pad may include a first connector and a second connector. The first connector is designed to connect to a third connector located on the wearing body, corresponding to the position of the first connector. The second connector may be adapted to connect to a fourth connector positioned on the control box, which corresponds to the location of the second connector. The second connector is located on the connecting portion of the treatment pad.

The first and second connectors may be configured to magnetically connect with the third and fourth connectors, respectively, allowing for easy attachment and detachment.

In one aspect, the second connector may be a first magnetic electrode, while the fourth connector may be a second magnetic electrode. These components are adapted to magnetically connect, establishing an electrical connection between the treatment pad and the control box.

Further, the treatment pad may consist of a light transmission layer, a light source layer, and a lining layer. The light transmission layer may be connected to the lining layer, with the light source layer positioned in between, facilitating the emission of light for phototherapy treatment.

The treatment pad may also include a plurality of notches arranged along the length. The plurality of notches has a width that gradually increases in a direction perpendicular to the length of the treatment pad, allowing for flexibility and adaptability to various head shapes.

The control box may have a housing that contains an energy storage element and a control panel. The control panel is electrically connected to the energy storage element, allowing for the operation of the wearable device.

In one aspect, the housing of the control box may include a box body that accommodates the energy storage element and the control panel, with a sealing plate covering the box body. This sealing plate may be adapted to detachably connect with the connecting portion of the treatment pad.

In another aspect, the control box may be mounted on an outer surface of the wearing body. The control box can be detachably connected to either the wearing body or the treatment pad using a connecting seat, providing an electrical connection to operate the treatment pad.

The control box may have a first end that connects to the wearing body and a second end that is designed to establish a detachable electrical connection with the treatment pad. The first end of the control box may include a first connector, designed to connect with a corresponding second connector on the wearing body, securing the control box to the wearing body's surface. The second end of the control box may include a third connector, which connects to a fourth connector located on the treatment pad, facilitating an electrical connection while securing the control box to the wearing body.

Further, the connecting seat may be detachably connected to the wearing body and connect to the second end of the control box, which has a third connector. The third connector is designed to engage with a fourth connector located on the treatment pad, forming an electrical connection while positioned on the wearing body.

The connecting seat may include two parallel connecting pieces configured to attach to the third connector of the control box. The connecting pieces are designed to accommodate a portion of the treatment pad having the fourth connector, establishing an electrical connection with the third connector.

Furthermore, the control box may include a housing that includes an energy storage element and a control panel. The control panel is electrically connected to the energy storage element for managing the device's operation.

The housing of the control box may include a box body containing the control panel and the energy storage element, with a sealing plate covering the box body for protection. The control box may contain a plurality of boxes arranged along the length of the control box, each designed to accommodate a plurality of energy storage elements, enhancing the power supply for the device.

Additionally, the treatment pad may include a central pad with a plurality of extension pads that extend radially in a circular direction. A first notch may be formed between adjacent extension pads, with the width of this notch gradually increasing away from the central pad. Each extension pad may have a second notch, where the width increases from the bottom of the notch to its opening direction.

In one aspect, the wearing body may include a connecting port formed on the wearing body, with the control box positioned at an open end of the connecting port. This arrangement allows for adjustment of the wearing body to fit the user's head.

In another aspect, the wearing body may feature a connecting port with a connecting band at an open end. The connecting band is designed to adjust the size of the wearing body. The control box may be positioned on an outer side of the connecting band, and attached to the treatment pad for secure operation.

### BRIEF DESCRIPTION OF DRAWINGS

Other features and advantages of the invention will become apparent when reading the detailed description given below, purely by way of example and in a non-limitative manner, referring to the following figures: FIG. 1 shows a perspective view of a wearable device for promoting hair growth through phototherapy in accordance with the present invention;

FIG. 2 shows an internal view of a wearable device for promoting hair growth through phototherapy in accordance with the present invention;

FIG. 3 shows an exploded view of a treatment pad and a control box of the wearable device for promoting hair growth through phototherapy;

FIG. 4 shows an internal view of a wearable device for promoting hair growth through phototherapy;

FIG. 5 shows an external view of the wearable device for promoting hair growth through phototherapy after assembling all the elements into a wearing body;

FIG. 6 shows a perspective view of the treatment pad and the control box of a wearable device illustrating the construction of the treatment pad;

FIG. 7 shows a schematic view of the wearable device for promoting hair growth through phototherapy in accordance with a second embodiment of the present invention;

FIG. 8a-8b shows a top view and a side view of a control box in accordance with the embodiment shown in FIG. 7;

FIG. 9 shows a perspective view of a treatment pad in accordance with the embodiment shown in FIG. 7;

FIG. 10 shows an exploded view of the treatment pad and a control box in accordance with the second embodiment;

FIG. 11 shows an exploded view of a control box in accordance with one of the aspects of the embodiment shown in FIG. 7;

FIG. 12 shows an exploded view of a treatment pad in accordance with the embodiment shown in FIG. 7;

FIG. 13 shows a front view of a treatment pad illustrating various portions of the treatment pad in accordance with the second embodiment shown in FIG. 7; and

FIG. 14 shows an external view of the wearable device for promoting hair growth through phototherapy in accordance with the embodiment shown in FIG. 7.

### DETAILED DESCRIPTION

An embodiment of this invention, illustrating its features, will now be described in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The disclosed embodiments are merely exemplary of the invention, which may be embodied in various forms.

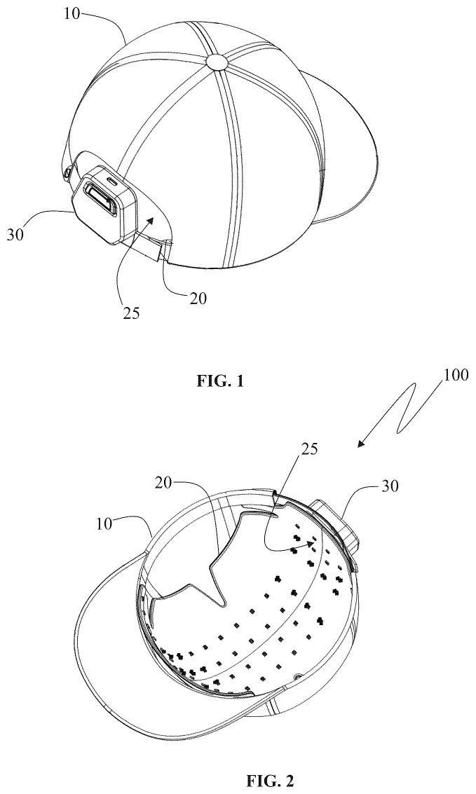

Referring now to FIGS. 1 and 2, a wearable device (100) for promoting hair growth through phototherapy in accordance with the present invention is illustrated. The wearable device (100) includes a wearing body (10) to be used to wear on a head of a user, a treatment pad (20) for emitting light to promote hair growth, and a control box (30) to operate the treatment pad (20).

The treatment pad (20) is arranged within the wearing body (10) configuring an inner shape of the wearing body (10) and is detachably connected with an inner side surface of the wearing body (10). The treatment pad (20) includes a connecting portion (25), especially, the connecting portion (25) is on a side of the treatment pad (20) that is in contact with the wearing body (10). The connecting portion (25) extends beyond a portion of the wearing body (10) and is detachably connected with the control box (30), specifically, the connecting portion (25) provides a surface to attach the control box (30) to the treatment pad (20) for establishing an electrical connection with the treatment pad (20).

The wearing body (10) and the treatment pad (20) are detachably connected to facilitate the treatment pad (20) to be disassembled from the wearing body (10) to facilitate the cleaning of the wearing body (10) and the treatment pad (20). The treatment pad (20) is detachably connected with the control box (30) and is exposed on an outer surface of the wearing body (10). The control box (30) is adapted to disassemble from the treatment pad (20) separately to prevent damage to the control box (30). In the preferred way, the wearing body (10) and the treatment pad (20) are used together to provide light therapy for hair growth. In another aspect of the present invention, the wearing body (10) can also be worn separately according to the user's needs.

In the present embodiment, the wearing body (100 is a cap. In another embodiment, the wearing body (10) can be selected as a hat, such as a baseball cap, a sunshade, etc., not limited to that, the wearing body (10) can also be selected as the shell of a hair growth instrument, and the hair growth instrument such as a hair growth treatment instrument, a laser hair growth instrument, etc.

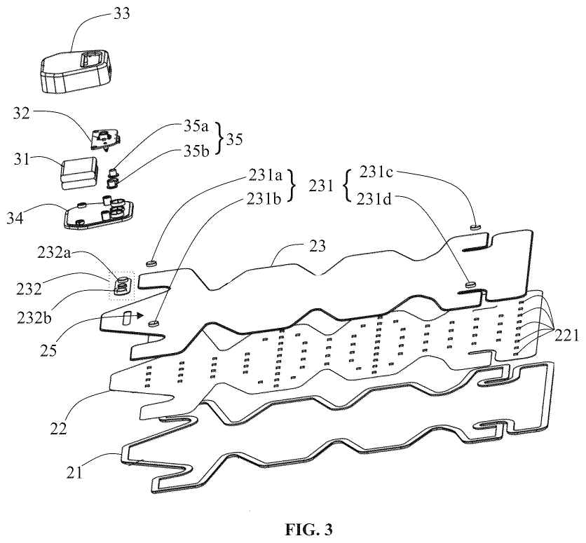

Referring now to FIG. 3, the treatment pad (20) comprises a light transmission layer (21), a light source layer (22) and a lining layer (23). The light transmission layer (21) is connected with the lining layer (23), and the light source layer (22) is located between the lining layer (23) and the light transmission layer (21). The light transmission layer (21) is connected with the lining layer (23) through the light source layer (22) arranged therebetween. The light transmission layer (21) is a transparent silicone sheet, the lining layer (23) is a silica gel sheet, and the light source layer (22) is a flexible LED light board. The light source layer (22) is provided with a plurality of light-emitting elements (221) for emitting red light. The light transmission layer (23) is made of white opaque silicone material, which allows the light-emitting element (221) of the light source layer (22) to emit red light. The red light has a germinating effect. In another embodiment, the light-emitting element (221) may be an LED lamp, a neon lamp, or other lamp that can emit red light/.

In the present embodiment, the light transmission layer (21), the light source layer (22) and the lining layer (23) are pressed together to form an integrated treatment pad (20) by hot pressing. In other embodiments, the light transmission layer (21), the light source layer (22) and the lining layer (23) may also be pasted together or injection molding to form an integrated treatment pad (20).

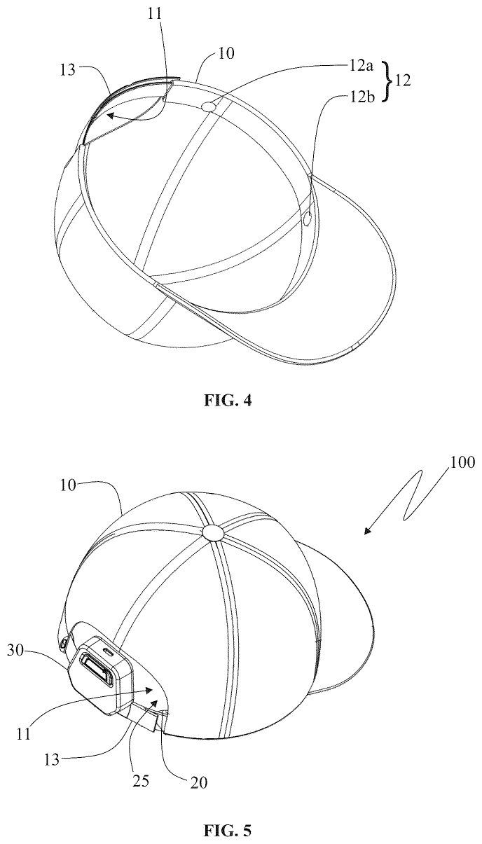

Referring now to FIGS. 3 and 4, the treatment pad (20) includes a first connector (231) and a second connector (232). The first connector (231) is adapted to detachably connect with a third connector (12) arranged on the wearing body (10) corresponding to a position of the first connector (231) to connect the treatment pad (20) to the wearing body (10). The second connector (232) is adapted to detachably connect with a fourth connector (35) arranged on the control box (30) corresponding to a position of the second connector (232) to attach the control box (30) to the treatment pad (20). Specifically, the second connector (232) is arranged on the connecting portion (25) of the treatment pad (20).

Further, in the present embodiment, the lining layer (23) includes a plurality of first connectors (231a, 231b, 231c, 231d) that are arranged at intervals, and a plurality of third connectors (12a, 12b) are arranged on the wearing body (10). In the present embodiment, the plurality of first connectors (231a, 231b, 231c, 231d) arranged on the lining layer (23) are four, and each first connector of the plurality of first connectors (231a, 231b, 231c, 231d) connects with the corresponding third connector (12) of the plurality of the third connectors (12a, 12b) (only two third connectors are shown). Further, a plurality of second connectors (232a, 232b) are arranged on the lining layer (23) of the treatment pad (20) at intervals, and a plurality of fourth connectors (35a, 35b) are arranged on the control box (30). Each second connector (232) of the plurality of second connectors (232a, 232b) connects with the corresponding fourth connector (35) of the plurality of fourth connectors (35a, 35b).

In the present embodiment of the invention, the first connector (231) and the third connector (12) are magnetically connected with each other. For example, the first connector (231) is a magnet, and the third connector (12) is a magnet or an iron block. In another embodiment of the present application, the first connector (231) and the third connector (12) may have Velcro attachment. In another embodiment of the present application, the first connector (231) is connected with the third connector (12) through a snap-fit attachment. For example, the first connector (231) is a fastener, and the third connector (12) is a buckle positioned on the wearing body (10) to be matched with the fastener.

Further, the second connector (232) is magnetically connected with the fourth connector (35). For example, the second connector (232) is a magnet, and the fourth connector (35) is also a magnet adapted to connect to the second connector (232). In another embodiment of the present invention, the second connector (232) is a first magnetic electrode and the fourth connector (35) is a second magnetic electrode adapted to magnetically connect with each other to hold the control box (30) on the treatment pad (200 and also establish an electrical connection between the treatment pad (20) and the control box (30). Specifically, the first magnetic electrode is electrically connected with the light source layer (22) and also is electrically connected with the fourth connector (35) arranged on the control box (30) to establish the electrical connection between the treatment pad (20) and the control box (30).

The first connector (231) is matched with the third connector (12) to make a detachable connection between the treatment pad (20) and the wearing body (10), and the plurality of the second connector (232a, 232b) is matched with the fourth connector (35) to make the detachable connection between the control box (30) and the treatment pad (20). The detachable connection allows the disassembly and cleaning of the wearing body (10), the treatment pad (20), and the control box (30).

In an embodiment, if the second connector (232) and the fourth connector (35) are both selected as magnetic suction electrodes, the control box (30) and the treatment pad (20) are magnetically connected and adapted to electrically communicate through magnetic suction electrodes.

In another embodiment, the first connector (231) and the third connector (12) are both magnets, the first connector (231) is installed on the lining layer (23) of the treatment pad (20) and connects with the third connector (12) to hold the treatment pad (20) therein. The magnet on the treatment pad (20) is magnetically matched with the magnet on the wearing body (10), and the treatment pad (20) is magnetically connected to the wearing body (10) to hold the treatment pad (20) within the wearing body (10) and does not establish an electrical connection therebetween similar to the second connector (232) and the fourth connector (35). The magnetic attachment and detachment of the treatment pad (20) with the wearing body (10) allows the disassembly and cleaning of the treatment pad (20).

Referring now to FIG. 3, the control box (30) comprises a housing (not shown), an energy storage element (31) and a control panel (32) arranged in the housing. The housing comprises a box body (33) and a sealing plate (34). The control panel (32) is electrically connected with the energy storage element (31). The box body (33) has an inner chamber accommodating the energy storage element (31) and the control panel (32). The sealing plate (34) covers the box body (33) and is detachably connected with the connecting portion (25) of the treatment pad (20). Specifically, the second connector (232) of the treatment pad is connected to the sealing plate (34). In the present embodiment, the energy storage element (31) is a battery and is used for providing power to the control panel (32).

Referring now to FIG. 4, and 5, the wearing body (10) includes a connecting port (11) formed corresponding to the position of the connecting portion (25) in the wearing body (10). Specifically, the wearing body (10) includes a cut portion arranged on the edge of the wearing body (10), and a connecting band (13) arranged on the wearing body (10) located on the opening side of the cut portion for adjusting the size of the cut portion. The connecting band (13) encloses the wearing body (10) to form the connecting port (11) after assembling all the elements of the wearable device (100) to the wearing body (10). In the present embodiment, the connecting band (13) can be an elastic band.

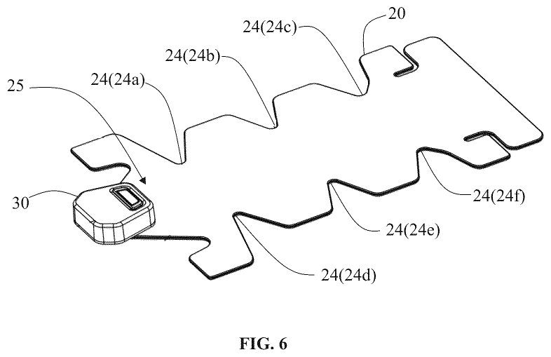

Referring now to FIG. 6, the treatment pad (20) includes a notch (24) specifically, a plurality of notches (24a, 24b, 24c, 24d, 24e, 24f). The plurality of notches (24a, 24b, 24c, 24d, 24e, 24f) is arranged along the length direction of the treatment pad (20), and the width of the plurality of notches (24a, 24b, 24c, 24d, 24e, 24f) gradually increasing in a direction perpendicular to the length of the treatment pad (20) up to a transverse end of the treatment pad (20). Specifically, the width of the plurality of notches (24a, 24b, 24c, 24d, 24e, 24f) gradually increases from the bottom of the notch (24) to the opening direction of the notch (24). The notch (24) is having V-shape. In the present embodiment, three V-shaped notches (24a, 24b, 24c, 24d, 24e, 24f) are arranged along the two sides of the width direction of the light transmission layer (21), the light source layer (22) and the lining layer (23). The notch (24) prevents the treatment pad (20) from wrinkling along with the bending of the treatment pad (20) when the treatment pad (20) is arranged into the wearing body (10).

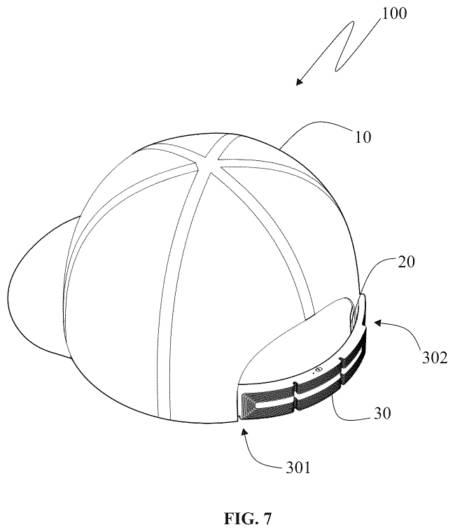

Referring now to FIG. 7, in a second embodiment of the present invention, the wearable device (100) comprises a wearing body (10), a treatment pad (20) and a control box (30).

The treatment pad (20) is arranged within the wearing body (10) configuring an inner shape of the wearing body (10) and is arranged on the inner side surface of the wearing body (10). The treatment pad (20) is used for emitting light to promote hair growth. The control box (30) is arranged on an outer side surface of the wearing body (10), and is detachably connected with the treatment pad (20) and/or the wearing body (10).

The control box (30) is arranged on the outer side surface of the wearing body (10), and is detachably connected with the treatment pad (20) and/or the wearing body (10), and the control box (30) is electrically connected with the treatment pad (20) and is used to operate the treatment pad (20).

In the present embodiment, the treatment pad (20) is detachably connected to the wearing body (10), and the control box (30) is detachably connected to the treatment pad (20) or the wearing body (10). Specifically, the control box (30) includes a first end (301) adapted to detachably connect to the wearing body (10), and a second end (302) adapted to detachably connect with the treatment pad (20) and establish an electrical connection when connected to the treatment pad (20).

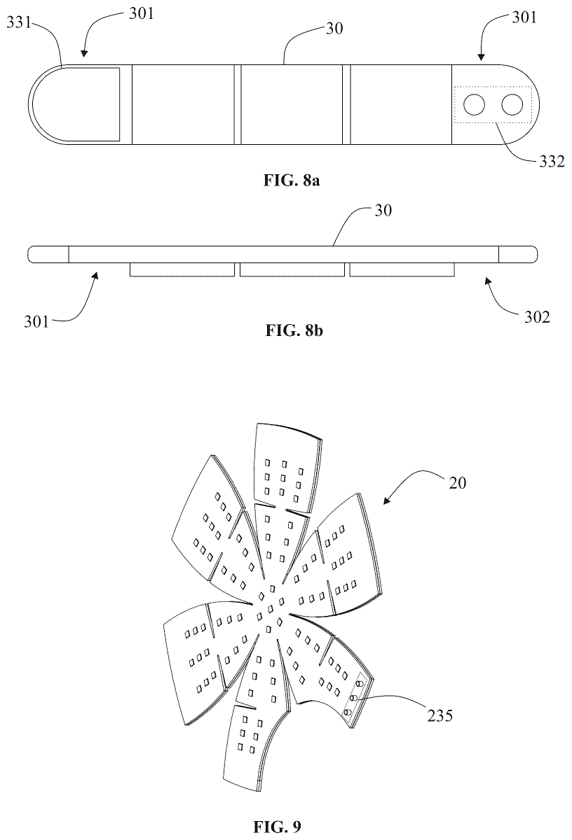

Referring to FIGS. 7, 8a, and 8b, the first end (301) of the control box (30) includes a first connector (331) to be connected with a second connector (not shown) arranged on the wearing body (10) to arrange the control box (30) on the surface of the wearing body (10). The second connector is arranged at the position corresponding to the first connector (331) adapted to connect with the first connector (331) detachably. Further, the second end (302) of the control box (30) includes a third connector (332) to be connected with a fourth connector (235) (shown in FIG. 9) arranged on the treatment pad (20) at the position corresponding to the third connector (332). Specifically, the fourth connector (235) is arranged on the light source layer (22) of the treatment pad (20) adapted to connect with the third connector (332). The third connector (332) is connected to the fourth connector (235) to arrange the control box (30) on the surface of the wearing body (10) and establish an electrical connection with the treatment pad (20).

Specifically, the first connector (331) is detachably connected with the second connector to attach the treatment pad (20) to the wearing body (10), and the third connector (332) is detachably connected with the fourth connector (235) to attach the control box (30) to the treatment pad (20) or the wearing body (10). In another embodiment of the present application, the control box (30) is only detachably connected with the wearing body (10), using the first connector (331) and the second connector. In another embodiment of the present application, the control box (30) is only detachably connected with the treatment pad (20), and only the third connector (332) and the fourth connector (235) are detachably connected with each other to arrange the control box (30) on the wearing body (10).

In one embodiment of the present application, the first connector (331) is magnetically connected with the second connector, wherein the first connector (331) and the second connector are magnets, or one of the first connectors (331) and the second connector is a magnet and the other is an iron block. Wherein, the number of the first connector (331) and the second connector is not limited, and can be appropriately set according to actual needs.

In another embodiment of the present application, the first connector (331) is a Velcro, and the second connector is also a Velcro to connect with each other. In another embodiment of the present application, the first connector (331) and the second connector are buckle adapted to connect with each other. In another embodiment of the present application, the first connector (331) and the second connector may also be detachably connected through fasteners such as screws.

In one embodiment of the present application, the third connector (332) is magnetically connected with the fourth connector (235), wherein the third connector (332) and the fourth connector (235) are magnets, or one of the third connector (332) or the fourth connector (235) is a magnet and the other is an iron block. In another embodiment of the present application, the fourth connector (235) is a first magnetic electrode electrically connected with the light source layer (22), and the third connector (332) is a second magnetic electrode magnetically matched with and electrically connected with the first magnetic electrode.

In the embodiment of the present application, the treatment pad (20) is detachably connected to the wearing body (10). In this way, the treatment pad (20) can also be removed from the wearing body (10), so that the treatment pad (20) and the wearing body (10) can be cleaned separately.

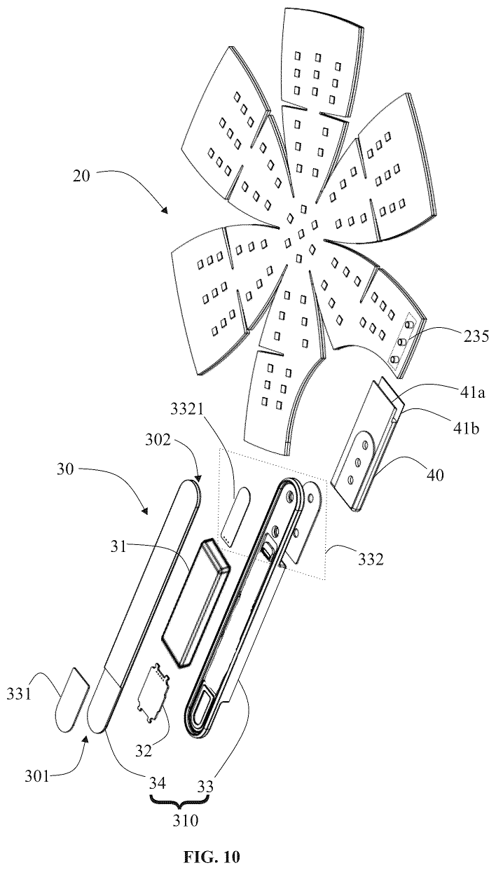

Referring to FIG. 10, the wearable device (100) includes a connecting seat (40) detachably connected with the edge of the wearing body (10) (not shown). The connecting seat (40) includes two connecting pieces (41a, 41b) arranged parallel to each other and configured to connect with the third connector (332) of the control box (30). The connecting pieces (41a, 41b) are connected to the wearing body (10). Further, the two connecting pieces (41a, 41b) are adapted to receive a portion of the treatment pad (20) having the fourth connector (235) establishing an electrical connection with the third connector (332).

Further, the control box (30) is located on one side of the connecting seat (40) which is away from the wearing body (10). The fourth connector (235) of the treatment pad (20) is adapted to be received by the two connecting pieces (41a, 41b). The fourth connector (235) receives the connecting pieces (41a, 41b) to contact with the third connector (332) of the control box (30). Specifically, the third connector (332) includes a connecting element (3321) which is in contact with the fourth connector (235) and one end of the connecting element (3321) is connected to the energy storage element (31) or the control unit (32) to make an electrical connected therebetween. Further, the connecting seat (40) is detachably connected to the wearing body (10), and the treatment pad (20) is detachably connected to the connecting seat (40). The connecting seat (40) is connected to the second end (302) of the control box (30) having the third connector (332) which is engageable with the fourth connector (235) to electrically connect with the control box (30) while being detachably attached to the wearing body (10).

The control box (30) includes a housing (310), an energy storage element (31), and a control panel (32) electrically connected with the energy storage element (31). Further, the housing (310) includes a box body (33) accommodating the energy storage element (31) and the control panel (32) therein, and a sealing plate (34) covering the box body (33).

The sealing plate (34) is located on one side of the control box (30) towards the wearing body (10), and the sealing plate (34) is detachably connected with the box body (33) to accommodate the components in the housing (310). Further, the first connector (331) is arranged on one side of the sealing plate (34) towards the wearing body (10).

The connecting seat (40) is fitted to one of the sides of the housing (310) that is separated from the wearing body (10), and the connecting seat (40) is located at one end of the housing (310). The side of the housing (310) that deviates from the wearing body (10) is used to expose the third connector (332). The connecting pad (40) comprises two connecting pieces (41a, 41b) arranged in parallel and at intervals, the treatment pad (20) is provided with the fourth connector (235) and one end is inserted between the two connecting pieces (41a, 41b), and the fourth connector (235) receives the connecting pieces (41a, 41b) to contact with the third connector (332), to establish an electrical connection between the treatment pad (20) and the control box (30).

The control box (30) has a flat long shape and includes the first end (301) adapted to detachably attach with the wearing body (10), and the second end (302) adapted to detachably attach with the treatment pad (20) to arrange the control box (30) on an outer surface of the wearing body (10). The first end (301) of the control box (30) is magnetically connected to the wearing body (10). In another embodiment, the first end (301) of the control box (30) is connected to the wearing body (10) through a snap-fit attachment. In another embodiment, the control box (30) is connected to the wearing body (10) through the Velcro attachment.

Further, the second end (302) of the control box (30) along its length direction is attached to the connecting seat (40), and is adapted to establish an electrical connection with the treatment pad (20) through the third connector (332).

It may be obvious for a person skilled in the art to attach the control box (30) and the wearing body (10) using other attachments. For example, the control box (30) is attached to the wearing body (10) using magnetic attachment at both ends along its length direction, or the control box (30) is attached to the wearing body (10) with both ends connected with buckles, or the two ends of the control box (30) along its length direction are provided with Velcro, and the wearing body (10) is also provided with the Velcro to attach with each other.

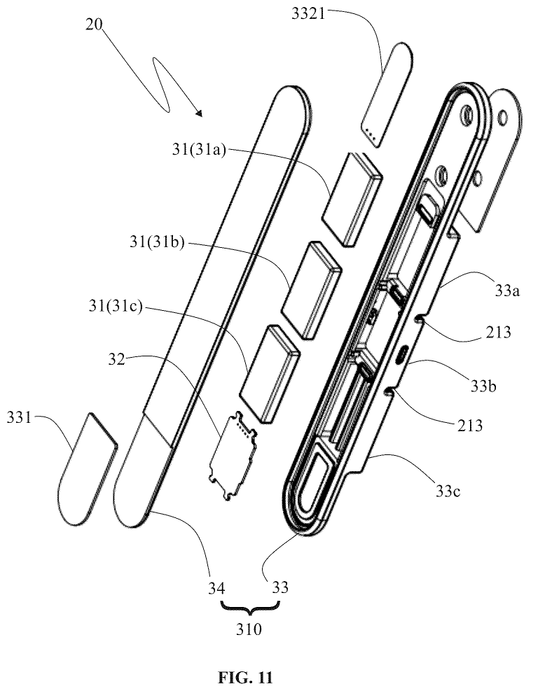

Referring to FIG. 11, in one of the aspects of the embodiment shown in FIG. 7, the control box (30) includes a plurality of energy storage elements (31a, 31b, 31c) arranged within the housing (310). The housing (310) is a flexible shell and includes a box body (33) having a plurality of boxes (33a, 33b, 33c) distributed at intervals along the length direction of the housing (310), a separation groove (213) formed between the adjacent two boxes (33a and 33b, or 33b and 33c), and the plurality of energy storage elements (31a, 31b, 31c) respectively arranged in the corresponding boxes (33a, 33b, 33c) one by one. The plurality of energy storage elements (31a, 31b, 31c) is stored within the plurality of boxes (33a, 33b, 33c) at the same time. When the control box (30) is attached to the treatment pad (20) and/or is arranged on the wearing body (10), the separation groove (213) formed between the plurality of boxes (33a and 33b, or 33b and 33c), allows the control box (30) to bend and avoid the interference of the adjacent box (33a or 33b or 33c).

In this way, the weight of the control box (30) is distributed on the wearing body (10), and can reduce the force displacement on the treatment pad (20) compared with the that the control box (30) is located in the wearing body (10) and is fixed on the treatment pad (20).

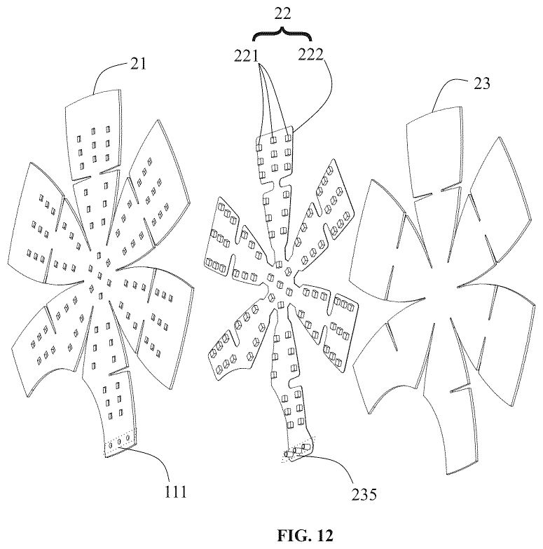

Referring now to FIG. 12, the treatment pad (20) comprises a light transmission layer (21), a light source layer (22) and a lining layer (23). The light transmission layer (21) is connected with the lining layer (23), and the light source layer (22) is located between the lining layer (23) and the light transmission layer (21). The light transmission layer (21) is connected with the lining layer (23) through the light source layer (22) arranged therebetween. Specifically, the light transmission layer (21) can be a transparent silicone sheet, which provides good light transmittance and has a good touch to improve the user experience. The light source layer (22) is a flexible LED light board, which comprises a flexible substrate (222) and a plurality of light-emitting elements (221) arranged on the flexible substrate (222), the light-emitting elements (221) are used for emitting red light, and the light source layer (22) can still maintain the stable luminescence of the light-emitting elements (221) in the bending process. The bottom layer (13) is a silicone sheet, which can maintain a good fit with the wearing body (10) and provide a good touch.

Further, the light transmission layer (21) includes a light outlet hole formed in the position corresponding to the light-emitting elements (221). The light-emitting elements (221) are placed in the light outlet hole in conjunction to arrange the light transmission layer (21) and the flexible substrate (222) with each other. Further, the fourth connector (235) is arranged on the light source layer (22), specifically, the light transmission layer (21) is provided with a through hole (111) at the corresponding position of the fourth connector (235), and the fourth connector (235) penetrates through the through hole (111) to contact with the third connector (332).

In alternative embodiments (not shown) of the present application, the light source layer (22) may also include a flexible substrate (222) and a neon lamp arranged on the flexible substrate (222) or other lamps that can emit red light, and no restriction is made herein.

In some embodiments of the present application, the light transmission layer (21), the light source layer (22) and the lining layer (23) are pressed, pasted and connected or injected by hot pressing to form a whole, so as to improve the integrity of the treatment pad (20) and avoid affecting the luminous stability of the treatment pad (20) due to the relative movement between the layers.

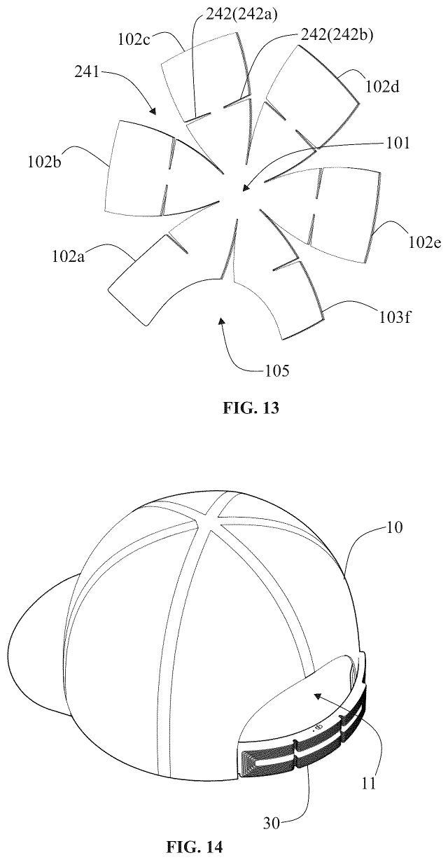

Referring now to FIG. 13, the treatment pad (20) comprises a central pad (101) and a plurality of extension pads (102a, 102b, 102c, 102d, 102e, 102f) radially extending away from the central pad (101). Further, the plurality of extension pads (102a, 102b, 102c, 102d, 102e, 102f) includes a first notch (241) formed between the two adjacent extension pads (102a, 102b, 102c, 102d, 102e, 102f), and the width of the first notch (241) gradually increases along the direction away from the central pad (101). The extension pad (102) is provided with a second notch (242) on a side adjacent to the first notch (241), and the width of the second notch (242) gradually increases from the bottom of the second notch (242) to the opening direction of the second notch (242).

In this embodiment, the extension pad (102) is provided with the second notch (242) on both sides of the first notch (241), so that the bending deformation of the extension pad (102) is relatively balanced and the flatness is improved. Specifically, the two second notches (242a, 242b) are arranged on each extension pad (102).

It is obvious that the central pad (101) and the plurality of extension pads (102a, 102b, 102c, 102d, 102e, 102f) are formed by arranging the light transmission layer (21), the light source layer (22) and the lining layer (23) over each other (as shown in FIG. 12).

Referring now to FIG. 14, in the embodiment of the present application, the wearing body (10) includes a cut portion arranged at the edge of the wearing body (10), and a connecting port (11) formed on the wearing body (10) due to the arrangement of the control box (30). The control box (30) is arranged at an open end of the cut portion and is used for adjusting the size of the cut portion. The open end is a portion of the cut portion, at a peripheral edge of the wearing body (10). In this configuration, the control box (30) is adapted to adjust the size of the wearing body (10) according to the size of the head of the user. The control box (30) and the wearing body (10) enclose the connecting port (11), and the circumference of the edge of the wearing body (10) is adjustable to adapt to the head circumference of different users.

In order to avoid exposing the treatment pad (20), an avoidance gap (105) (shown in FIG. 13) is arranged on the extension pad (102), especially on two extension pads (102a, 102f) corresponding to the cut portion of the wearing body (10), so as to improve the aesthetics while protecting the treatment pad (20). It is understood that in other embodiments of the present application, the cut portion may also be omitted, and correspondingly, the avoidance gap (105) may also be omitted, and no restriction is made herein.

In another embodiment (not shown), the wearing body (10) includes a connecting port (11) formed on the wearing body (10), having a connecting band (13) arranged at an open end of the connecting port (11). The connecting band (13) is adapted to adjust the size of the wearing body (10) according to the size of the user's head. In this embodiment, the control box (30) is arranged on an outer side of the connecting band (13) and attached to the treatment pad (20).

Further, in an embodiment (not shown), the control box (30) includes a power button arranged one of the sides to turn On or Off the light emitting from the treatment pad (20). Furthermore, the control box (30) may include a battery indicator to show the battery level of the energy storage element (31). Moreover, the control box may include a charging port (not shown) arranged on one of the sides of the control box, connected to recharge the energy storage element (31).

The wearable device (100) is provided to dissipate the heat away from the scalp by arranging the control box (30) on the outer side surface of the wearing body (10), thereby reducing the potential safety hazard when charging and heating. At the same time, the control box (30) is detachably connected with the treatment pad (20) and/or the wearing body (10) to provide convenience to be disassembled from the wearing body (10), and the control box (30) is also convenient for the treatment pad (20) to be disassembled. The treatment pad (20) and the wearing body (10) are further convenient for cleaning, the damage that the control box (30) may be caused by cleaning is avoided simultaneously the phototherapy hair growth equipment is inconvenient to disassemble the control box (30), and the control box (30) is charged and heats up with the problem that there is a potential safety hazard.

The foregoing descriptions of specific embodiments of the present invention have been presented for purposes of illustration and description. They are not intended to be exhaustive or to limit the present invention to the precise forms disclosed, and obviously, many modifications and variations are possible in light of the above teaching. The embodiments were chosen and described in order to explain the principles of the present invention best and its practical application, thereby enabling others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the scope of the claims of the present invention.

1. A wearable device (100) for promoting hair growth through phototherapy comprising:
a wearing body (10) to be used to wear on a head of a user;
a treatment pad (20) arranged within the wearing body (10) configuring an inner shape of the wearing body (10), the treatment pad (20) is adapted to emit light on the head of the user;
a control box (30) attachable to the wearing body (10), wherein the treatment pad (20) has a connecting portion (25), the connecting portion (25) extends beyond a portion of the wearing body (10) for providing a surface to attach the control box (30) to the treatment pad (20) for establishing an electrical connection with the treatment pad (20);
wherein the treatment pad (20) includes a first connector (231) and a second connector (232), the first connector (231) is adapted to connect with a third connector (12) arranged on the wearing body (10) corresponding to a position of the first connector (231), and the second connector (232) is adapted to connect with a fourth connector (35) arranged on the control box (30) corresponding to a position of the second connector (232), wherein the second connector (232) is arranged on the connecting portion (25) of the treatment pad (20);
wherein the second connector (232) is a first magnetic electrode and the fourth connector (35) is a second magnetic electrode adapted to magnetically connect and electrically communicate with each other through the first magnetic electrode and the second magnetic electrode for establishing an electrical connection between the treatment pad (20) and the control box (30) and for holding the control box (30) on the treatment pad (20).

2. (canceled)

3. (canceled)

4. (canceled)

5. The wearable device (100) as claimed in claim 1, wherein the treatment pad (20) includes a light transmission layer (21), a light source layer (22) and a lining layer (23), wherein the light transmission layer (21) is a transparent silicone sheet, the lining layer (23) is a silica gel sheet, arranged to protect the light source layer (22) therebetween, wherein the light source layer (22) is a flexible LED light board to transmit light through the light transmission layer (21) to the head of the user.

6. The wearable device (100) as claimed in claim 1, wherein the treatment pad (20) has a plurality of notches (24a, 24b, 24c, 24d, 24e, 24f), the plurality of notches (24a, 24b, 24c, 24d, 24e, 24f) are arranged along a length of the treatment pad (20), wherein the plurality of notches (24a, 24b, 24c, 24d, 24e, 24f) has a width gradually increasing in a direction perpendicular to the length of the treatment pad (20) up to a transverse end of the treatment pad (20).

7. The wearable device (100) as claimed in claim 1, wherein the control box (30) includes a housing accommodating an energy storage element (31) and a control panel (32) therein, wherein the control panel (32) is electrically connected with the energy storage element (31).

8. The wearable device (100) as claimed in claim 7, wherein the housing comprises a box body (33) accommodating the energy storage element (31) and the control panel (32) therein, and a sealing plate (34) covering the box body (33), the sealing plate (34) is adapted to connect with a connecting portion (25) detachably.

9. A wearable device (100) for promoting hair growth through phototherapy comprising:
a wearing body (10) to be used to wear on a head of a user;
a treatment pad (20) arranged within the wearing body (10) configuring an inner shape of the wearing body (10), the treatment pad (20) is adapted to emit light on the head of the user;
a control box (30) arranged on an outer surface of the wearing body (10), wherein the control box (30) is detachably connected to the wearing body (10) or the treatment pad (20) through a connecting seat (40), establishing an electrical connection with the treatment pad (20) to operate the treatment pad (20);
wherein the wearing body (10) includes a connecting port (11) formed on the wearing body (10), and the control box (30) is arranged at an open end of the connecting port (11), the control box (30) is adapted to adjust the size of the wearing body (10) according to the size of the user's head.

10. The wearable device (100) as claimed in claim 9, wherein the control box (30) includes a first end (301) adapted to detachably connect with the wearing body (10), and a second end (302) adapted to detachably connect with the treatment pad (20) and establishes an electrical connection when connected to the treatment pad (20).

11. The wearable device (100) as claimed in claim 10, wherein the first end (301) of the control box (30) includes a first connector (331) to be connected with a second connector arranged on the wearing body (10) to arrange the control box (30) on the surface of the wearing body (10).

12. The wearable device (100) as claimed in claim 10, wherein the second end (302) of the control box (30) includes a third connector (332) to be connected with a fourth connector (235) arranged on the treatment pad (20) to arrange the control box (30) on the surface of the wearing body (10) and establish an electrical connection with the treatment pad (20).

13. The wearable device (100) as claimed in claim 9, wherein the connecting seat (40) is detachably attached to the wearing body (10) and is connected to a second end (302) of the control box (30) having a third connector (332), engageable with a fourth connector (235) arranged on the treatment pad (20) to electrically connect with the control box (30) while being arranged on the wearing body (10).

14. The wearable device (100) as claimed in claim 13, wherein the connecting seat (40) includes two connecting pieces (41a, 41b) arranged parallel to each other and configured to connect with the third connector (332) of the control box (30), the two connecting pieces (41a, 41b) are adapted to receive a portion of the treatment pad (20) having the fourth connector (235) to establish an electrical connection with the third connector (332).

15. The wearable device (100) as claimed in claim 9, wherein the control box (30) includes a housing (310), an energy storage element (31), and a control panel (32) electrically connected with the energy storage element (31).

16. The wearable device (100) as claimed in claim 15, wherein the housing (310) includes a box body (33) accommodating the control panel (32) and the energy storage element (31) therein, and a sealing plate (34) covering the box body (33).

17. The wearable device (100) as claimed in claim 9, wherein the control box (30) includes a plurality of boxes (33a, 33b, 33c) arranged along a length direction of the control box (30) to accommodate a plurality of energy storage elements therein.

18. The wearable device (100) as claimed in claim 9, wherein the treatment pad (20) comprises a central pad (101) and a plurality of extension pads (102a, 102b, 102c, 102d, 102e, 102f) radially extending away from the central pad (101), the treatment pad (20) includes a first notch (241) formed between two adjacent extension pads (102), and the width of the first notch (241) gradually increases along the direction away from the central pad (101); the plurality of extension pads (102a, 102b, 102c, 102d, 102e, 102f) are provided with a second notch (242) on a side adjacent to the first notch (241), the width of the second notch (242) gradually increases from the bottom of the second notch (242) to the opening direction of the second notch (242).

19. (canceled)

20. The wearable device (100) as claimed in claim 9, wherein the wearing body (10) includes a connecting port (11) formed on the wearing body (10), having a connecting band (13) arranged at an open end of the connecting port (11), the connecting band (13) is adapted to adjust the size of the wearing body (10) according to the user head, wherein the control box (30) is arranged on an outer side of the connecting band (13) and attached to the treatment pad (20).
